(19) United States
(12) Patent Application Publication
(10) Pub. No.: US 2026/0335556 A1
(43) Pub. Date: Oct. 29, 2026

(54) DYE SOLUTION, SYRINGE AND CANNULA PACKAGE FOR SELECTIVE DELIVERY TO OPHTHALMIC TARGET TISSUE

(71) Applicants: David Mordaunt, Los Gatos, CA (US); Alexander Mordaunt, Los Gatos, CA (US)

(72) Inventors: David Mordaunt, Los Gatos, CA (US); Alexander Mordaunt, Los Gatos, CA (US)

(21) Appl. No.: 19/656,234

(22) Filed: Apr. 23, 2026

Related U.S. Application Data
(60) Provisional application No. 63/793,840, filed on Apr. 24, 2025.

(51) Int. Cl. A61M 5/31

(52) U.S. Cl. CPC A61M 5/3134; A61M 2205/584; A61M 2210/0612

(57) ABSTRACT

Devices comprise a syringe, a Trypan Blue dye solution disposed in the syringe, and a cannula attached to or attachable to the syringe for which a combination of syringe volume capacity, blue dye solution concentration and volume, and cannula inner dimensions (e.g., diameter or average diameter) synergistically interact to allow for controlled application of the blue dye solution to stain ophthalmic tissue without the solution appreciably dispersing to a large portion of the eye. Related methods comprise using such a device to apply the blue dye solution to the ophthalmic tissue at a rate of less than or equal to about 0.05 ml/s. The blue dye solution may advantageously be applied to the ophthalmic tissue by moving a tip of the cannula in a zigzag pattern along or the ophthalmic tissue.

### CROSS REFERENCE TO RELATED APPLICATIONS

This patent application claims benefit of priority to U.S. Provisional Patent Application 63/793,840 filed 24 Apr. 2025, which is incorporated herein by reference in its entirety.

### FIELD OF THE INVENTION

The invention relates generally to aqueous dye solutions and syringe and cannula packages employed in ophthalmologic surgeries to selectively stain or color acellular membranes.

### BACKGROUND

Trypan Blue dye-based aqueous solutions are employed in ophthalmologic surgeries to stain or color acellular membranes. Within the context of this invention and in existing literature, the terms “dye” and “stain” are used interchangeably and are considered synonymous, referring to the application of dye to impart color to tissue. Certain dyes possess selective properties, enabling them to exclusively stain acellular tissue, such as non-living cells or membranes.

In cataract surgeries, Trypan Blue dye is employed to enhance the visibility of the anterior capsule, as first described by Melles in 1999. More recently, Mordaunt in 2009 (and associated patents) has used Trypan Blue to dye the anterior capsule before applying a laser to create a capsulotomy.

In vitreoretinal surgeries Teba 2003, described using Trypan Blue solutions to selectively stain the internal limiting membrane (ILM) and epiretinal membrane (ERM). Ahmad 2014 found that ILMs were more effectively stained with an alternative dye Brilliant Blue G. There are now some ophthalmic dye products with Brilliant Blue G and Trypan Blue combinations, or solutions of Brilliant blue without Trypan Blue, or Trypan Blue without Brilliant Blue.

As far as the inventors are aware, all blue dye solutions in ophthalmology are supplied in prefilled syringes with a capacity of 2 ml or larger-often with the volume of the solution being 0.5 ml or larger. Further these dye solutions and syringes are all provided with 27G or greater diameter cannula.

### REFERENCES CITED ABOVE OR OTHERWISE RELEVANT

Melles de Waard P W, Pameyer J H, Houdijn Beekhuis W. Trypan Blue capsule staining to visualize the capsulorhexis in cataract surgery. G. R. Melles et al., J Cataract Refract Surg, 1999; vol. 25, pp. 7-9.
Coroneo 1999, U.S. Pat No. 6,372,449B1 Ophthalmic Methods and Uses.
Mordaunt 2009, Patent US2009088734A1 Thermal Separation of Tissue and associated patents.
Mordaunt 2022, Patent US20220175971A1 Dye Enhanced Visualization of Cataract Surgery.
Teba F A, Mohr A, Eckardt C, Wong D, Kusaka S, Joondeph B C, Feron E J, Stalmans P, Van Overdam K, Melles G R. Trypan Blue staining in vitreoretinal surgery. Ophthalmology. 2003; vol 110, pp 2409-2412.
Ahmad C N, Shaheer M. Comparison of Brilliant Blue G and Trypan Blue during Vitrectomy for Macular Hole Surgery. Pakistan Journal of Ophthalmology, 2014; vol. 30, pp 227-231.
Packard R. Physico-chemical Characteristics of a Microfiltered 0.4% Trypan Blue Solution When Applied to the Anterior Capsule. J. Cataract Refract. Surg. 2025 Accepted.

### SUMMARY

This specification discloses devices comprising a syringe, a blue (e.g., Trypan Blue) dye solution disposed in the syringe, and a cannula attached to or attachable to the syringe for which a combination of syringe volume capacity, blue dye solution concentration and volume, and cannula inner dimensions (e.g., diameter or average diameter) synergistically interact to allow for controlled application of the blue dye solution to stain ophthalmic tissue without the blue dye solution appreciably dispersing to a large portion of the eye. The advantages of and synergistic features of the devices are described and characterized in more detail in the following specification and claims.

This application also discloses related methods that comprise using such a device to apply the blue (e.g., Trypan Blue) dye solution to the ophthalmic tissue at a rate of less than or equal to about 0.05 ml/s. The blue dye solution may advantageously be applied to the ophthalmic tissue by moving a tip of the cannula in a zigzag pattern along the ophthalmic tissue, as described and characterized in more detail in the following specification and claims.

These and other embodiments, features and advantages of the present invention will become more apparent to those skilled in the art when taken with reference to the following more detailed description of the invention in conjunction with the accompanying drawings that are first briefly described.

### BRIEF DESCRIPTION OF THE DRAWINGS

FIG. 1A illustrates an example device comprising Trypan Blue 0.2% (or greater) solution (11), a syringe (12) with about 1 ml capacity, a cannula (13) of 30 G, delivering a small stream (14) of Trypan Blue solution on to the anterior capsule (5) of an eye. This stream remains distinct and separate from the remaining fluid in the anterior chamber (1) of the eye. The cornea (2), the sclera (3), and the iris (4) are also illustrated.

FIG. 1B illustrates the same device at FIG. 1A, where a larger stream (15) of Trypan Blue solution is applied to the visible anterior capsule. The stream remains intact and coats the capsule.

FIG. 1C illustrates a device comprising Trypan Blue 0.2% (or greater) solution (11), a syringe (22) with 2 ml (or greater) capacity, a cannula (23) of 27 G, delivering a small stream (24) of Trypan Blue solution which disperses widely within the anterior chamber.

FIG. 1D illustrates the same device at FIG. 1C, where a larger stream (25) of Trypan Blue solution is fully mixed with the fluid in the anterior chamber.

FIG. 2A is a bird's-eye view black and white photograph of Trypan Blue solution 5 seconds after it has been applied to the base of a glass containing balanced salt solution (BSS) with a 30 G cannula from a syringe with a capacity of 1 ml. There are two regions: in Region 31 Trypan Blue solution of 0.3% was delivered under the BSS touching the glass surface. In Region 32 Trypan Blue solution of 0.06% was delivered under the BSS touching the glass surface. The Trypan Blue regions remain distinct and separate from the original BSS liquid in the glass.

FIG. 2B is a bird's-eye view black and white photograph of Trypan Blue solution of concentration 0.3%, 5 seconds after it has been applied to the base of a glass containing BSS with a 27 G cannula from a syringe with a capacity of 1 ml. The Trypan Blue solution swirls and rapidly mixes with the original fluid in the glass.

FIG. 2C is a bird's-eye view black and white photograph of Trypan Blue solution of concentration 0.3%, 15 seconds after it has been applied to the base of a glass containing BSS with a 27 G cannula from a syringe with a capacity of 1 ml. The Trypan Blue solution is now completely mixed with the original BSS liquid in the glass.

FIG. 3A is a side-view black and white photograph of a low-speed delivery of 0.3% Trypan Blue solution with a 30 G cannula from a syringe of 1 ml capacity into a 10 cm tall bottle of BSS. This shows the Trypan Blue solution stream remains intact and distinct from the original fluid in the bottle. Gravity influences the dye stream to descend and then coats the lower surface of the bottle.

FIG. 3B is a side-view black and white photograph of a high-speed delivery of 0.3% Trypan Blue solution with a 27 G cannula from a syringe of 1 ml capacity into a 10 cm tall bottle of BSS. This shows complete mixing of the Trypan Blue solution and the original BSS fluid in the bottle.

FIG. 4A is a bird's-eye view black and white photograph (as in FIG. 2A) of Trypan Blue solution 5 seconds after it has been applied to the base of a glass containing BSS with a 30 G cannula from a syringe with a capacity of 1 ml. There are two regions: in Region 31 Trypan Blue solution of 0.3% was delivered under the BSS touching the glass surface. In Region 32 Trypan Blue solution of 0.06% was delivered under the BSS touching the glass surface. The Trypan Blue remains distinct and separate from the original BSS liquid in the glass.

FIG. 4B is a side view black and white photograph of the glass in FIG. 4A, the Trypan Blue solution is not visible. It is a very thin layer of dye on the surface of the glass.

FIG. 4C is a bird's-eye view black and white photograph of the Trypan Blue solution applied as in FIG. 4A to the base of a glass containing BSS in two regions 31 and 32, 1 minute after the dye had been applied. Region 31 remains most unaffected with time, whereas the less concentrated dye in region 32 dissolves.

FIG. 4D is a birds'eye view black and white photograph of the Trypan Blue solution applied as in FIG. 4A and FIG. 4C to the base of a glass containing BSS in two regions 31 and 32, 5 hours after the dye had been applied. Region 31 remains only slightly lessened with time, whereas the less concentrated dye in region 32 has almost disappeared.

FIGS. 5A, 5B, 5C, and 5D show birds-eye and side view black and white photographs of a glass of BSS injected with a color solution through a 30 G cannula from a syringe of 1 ml capacity: FIG. 5A Trypan Blue dye solution, FIG. 5B Copper Sulphate solution (blue in color), FIG. 5C yellow food coloring dye solution, and FIG. 5D blue food coloring dye solution.

FIG. 6 shows a black and white photograph of a solution of 0.3% Trypan Blue following 0.1 micron filtration (non-polarized image shown).

FIGS. 7A, 7B, 7C, 7D, 7E, and 7F are anterior-posterior views of an eye and illustrate a “zigzag” method of applying the dye to the anterior chamber. In FIG. 7A the dotted line 74 describes the zigzag path of the cannular distill tip while dispensing the dye solution. FIGS. 7B-7F show the resultant dye stream layer that is formed on the anterior capsule as the cannula is moved along the “zigzag” path. The anterior capsule 71, the dilated iris 72, the delivery cannula 73, and the hydrophobic Trypan Blue solution 75 are also illustrated.

FIGS. 8A, 8B, 8C, 8D, 8E, and 8F are anterior-posterior views of the eye and illustrate the “zigzag” method of applying the dye to a target region of the retina. The dotted line 84 in FIG. 8A describes the zigzag path of the cannular distill tip while dispensing the dye solution. FIGS. 8B-8F show the resultant dye stream layer that is formed on the retina as the cannula is moved along the “zigzag” path. The region of interest in the retina 81, the retina 82, the delivery cannula 83, and the hydrophobic Trypan Blue solution 85 are also illustrated.

### DETAILED DESCRIPTION

The following detailed description should be read with reference to the drawings, in which identical reference numbers refer to like elements throughout the different figures. The drawings, which are not necessarily to scale, depict selective embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention. As used in this specification and the appended claims, the singular forms “a,” “an,” and “the” include plural referents unless the context clearly indicates otherwise.

As noted above, in conventional methods dye solutions are applied into the eye for anterior segment and vitreoretinal surgeries using prefilled syringes with a capacity of 2 ml or larger—often with the volume of the solution being 0.5 ml or larger, with the syringes provided with 27 G or greater diameter cannula. The inventors have observed challenges with the conventional approach. One of the main challenges of applying blue dye solutions with this configuration of devices is that delivery to the target tissue is not precisely controlled when using syringes with a capacity of 2 ml or greater and with cannulas of 27 G diameter or greater. There are three main issues:
1. By applying large volumes of dye solution, the target membrane is not selectively dyed and instead the dye is generally applied to the complete anterior or posterior chamber of the eye, flooding the whole chamber and non-target tissues are exposed to the dye.
2. Relatively high-speed delivery of the dye dilutes the dye making it less concentrated and in turn lessens the dye absorbed in the target tissue-resulting in a less intense stain.
3. Relatively high-speed delivery of the dye can disrupt sensitive cells causing absorption of the dye in these cells. This often happens in anterior segment surgery, where the corneal endothelium will be disrupted if exposed to dye delivered through a 27 G cannula from a 2 ml syringe. These disrupted endothelial cells can then absorb dye-which is an unwanted result in cataract surgery, as it can decrease visibility into the eye for surgery, can lead to increased corneal edema, and using a laser for anterior capsulotomy may leave a temporary mark on the endothelial layer.

The blue dye is for example an aqueous solution consisting of Trypan Blue and Brilliant Blue G dye combined, or it is Trypan Blue dye without Brilliant Blue G, or it is Brilliant Blue G without Trypan Blue. The solution is isotonic or has a tonicity near that of the native aqueous humor, i.e., about 304 mOsm/kg, and a pH of 6.8-7.6, to be close to that of the native aqueous humor.

A 27 G cannula is a thin, hollow tube used with a blunted distill tip and is used for various medical procedures. It has outside and internal diameters of about 413 and 210 microns respectively. By comparison, for a 30 G cannula the diameters are smaller, with the outside and internal diameters being about 312 and 159 microns respectively.

The combination of cannula and syringe capacity (or more specifically plunger diameter) regulates the delivery flow rate, given normal application pressures. The reduction in cannula internal cross-section area between 27 G and 30 G is about 50%, yet the flow rates are about 0.15 ml/s and 0.04 ml/s respectively when used with a 1 ml capacity syringe. Comparing 2 ml to 1 ml to 0.5 ml capacity syringes with 30 G cannula, rates are 0.01 ml/s, 0.04 ml/s and 0.12 ml/s respectively. So, the smaller volume syringe has a higher flow rate. The stroke length for the same volume of solution is 4-times with the 0.5 ml capacity syringe compared to 2 ml and is doubled for the 1 ml compared to 2 ml.

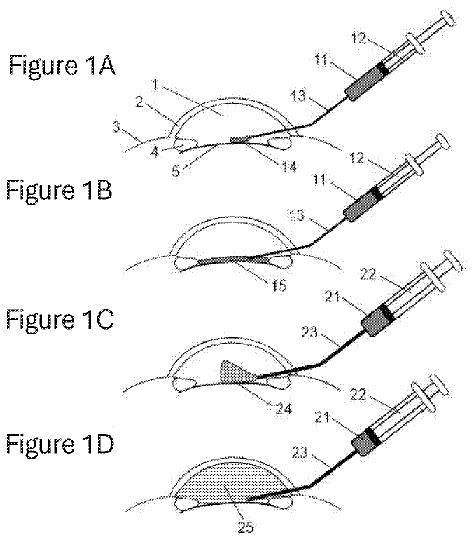

The inventors have observed that when a 30 G cannula is used in combination with a 1 ml capacity syringe, there is an ample control for the application of the blue dye solution to the target tissue. Significantly, the inventors have observed that for solutions that are injected into the anterior chamber of an eye with the following criteria:
a Trypan blue solution,
with a concentration greater than 0.2% by weight,
through a 30 G cannula
from a syringe with about a 1 ml capacity (plunger diameter of about 4.6 mm), the dye solution does not mix with the surrounding fluid, but instead remains a distinct and separate stream, as illustrated in FIG. 1A. As more fluid is injected it does not form a ball but is affected by gravity and descends, coating lower surfaces with a thin layer of concentrated dye solution as illustrated in FIG. 1B.

If the device configuration is changed to facilitate a higher flow rate, for example if a 27 G cannula is used, then the dye solution does not remain distinct but instead there is rapid mixing with the surrounding fluid, as illustrated in FIG. 1C. As the volume of dye solution is increased the anatomical chamber becomes flooded with a diluted Trypan blue solution, as illustrated in FIG. 1D.

The inventors have found that for solutions with a dye concentration of Trypan Blue about 0.2% (or more, by weight), the speed of delivery affects the delivered dye solution. At rates of about 0.05 ml/s or less, the dye solution does not mix with the surrounding fluid environment, rather it remains a separate distinct cohesive stream/pool/ball of dye solution that can be applied directly to the target tissue. It is also noted that this cohesive stream of dye solution has a specific gravity greater than water, and this cohesive stream sinks under the influence of gravity. The cohesivity limit of this blue dye solution is a delivery rate of about 0.05 ml/s or less.

If the rate of delivery is greater than 0.1 ml/s or greater, the dye solution does not remain as a distinct separate stream. Instead, the dye solution has major turbulent flow and the dye solution is mixed with the surrounding fluid, to such an extent that the anterior chamber volume can be totally mixed with the dye solution. There is no distinct dye cohesive stream, and the diffuse mixed solution is not influenced by gravity, rather it is mixed in the anatomical chamber as illustrated in FIGS. 1C and 1D. Gravity does not affect the resultant mixture of solutions.

Interestingly, using the smaller 0.5 ml capacity syringe has a tendency for the dye to flow at higher flow rates than the observed cohesivity limit and the dye was mixed with the surrounding fluid. The 2 ml capacity syringe has a slow delivery, but there was a lack of control given the minimal plunger draw length. The optimal device configuration that limits the maximum rate of delivery and keeps it within the range for the dye solution to remain separate and distinct is a solution volume of 0.3 ml or less, in a syringe with a capacity of about 1 ml, accompanied by a 30 G cannula.

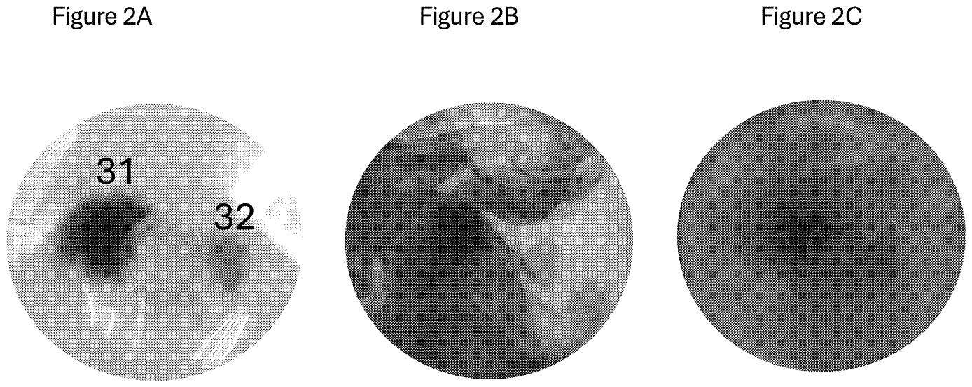

To investigate the effect of cannula size (and indirectly of the delivery rate) 0.3% concentration Trypan blue solution was injected into glasses containing BSS from a 1 ml capacity syringe through cannula of different sizes. FIG. 2A shows a bird's-eye view of an example in which a 30 G cannula was used to deliver the Trypan Blue under the BSS on to the glass near the base of the glass, forming region 31. The result is a blue intense layer of dye. In the example of FIG. 2B, the cannula was exchanged for a 27 G cannula, and the same amount of dye was delivered. The result is a swirl of the dye solution as it mixes with the surrounding fluid. In FIG. 2C, the result for the example of FIG. 2B is shown 15 seconds after delivery of the Trypan Blue solution, which is now completely mixed with the original fluid in the glass, making a highly diluted solution.

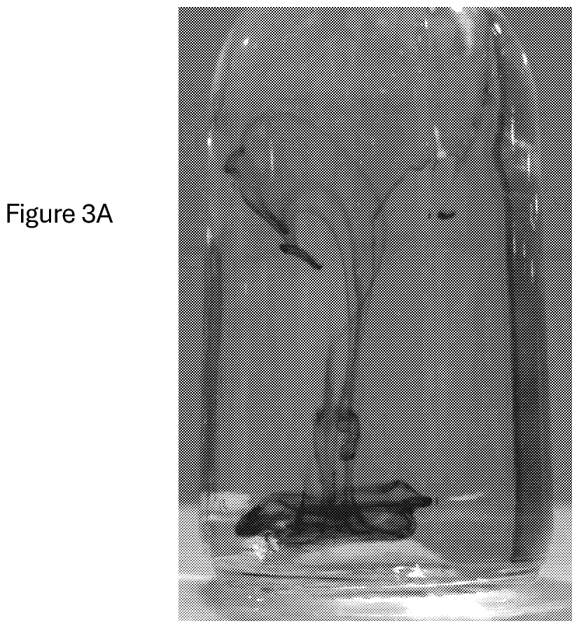

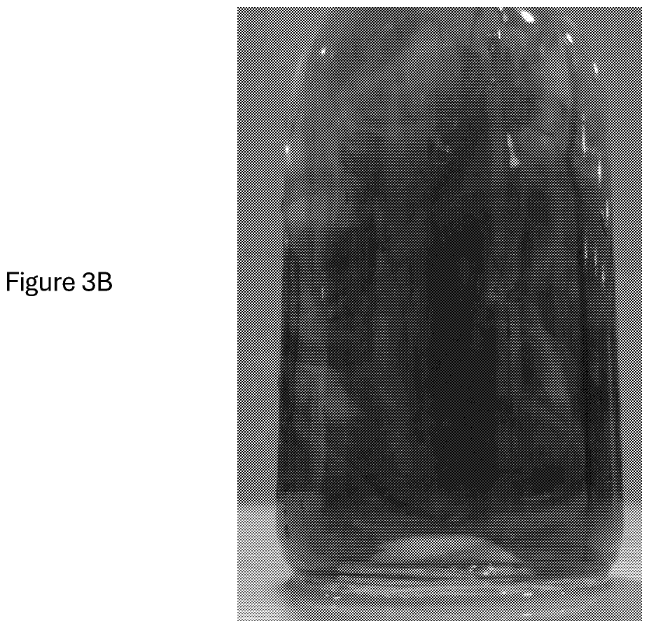

To further illustrate the effect of cannula size, FIG. 3A shows a photo of a 0.3% concentration Trypan Blue solution injected from a syringe with a capacity of 1 ml through a 30 G cannula into a 10 cm tall bottle of BSS. A cohesive stream of Trypan Blue solution is visible and distinct from the surrounding fluid. This stream descends to the base of the container and remains in concentrated form. If the area of the container allows, the dye will coat/cover the lower surface of the container. FIG. 3B shows a photo of 0.3% concentration Trypan Blue solution injected from a syringe with a capacity of 1 ml through a 27 G cannula. In this example the dye is delivered faster, it rapidly mixes with the surrounding liquid, and its concentration is diluted thus reducing the effective staining rate.

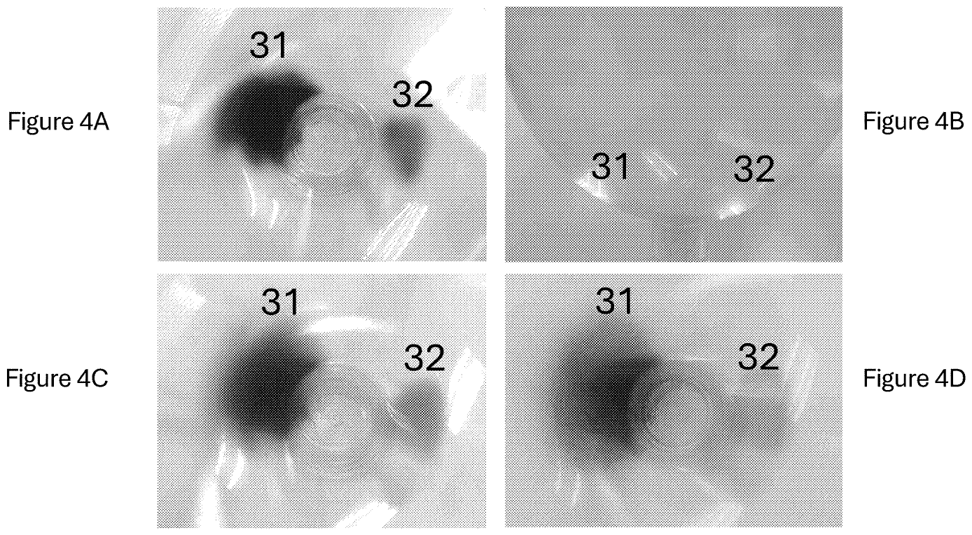

FIGS. 4A, 4B, 4C, and 4D show the stability of the resultant Trypan Blue layer resulting from using a 30 G cannula for two different concentrations. FIG. 4A shows two regions of Trypan Blue shortly after application. Region 31 is where 0.1 ml of 0.3% Trypan Blue solution was applied, and Region 32 is where 0.1 ml of 0.06% Trypan Blue solution was applied. It is obvious that Region 31 is a darker blue with a larger area and well-defined visible boundaries in comparison to Region 32. FIG. 4B shows that in both situations the Trypan blue is a very thin layer on the surface of the glass and is not easily visible in a side view.

As shown in FIG. 4C, within 15 seconds of injection, the dye in Region 32 readily dissolves leaving only a faint blue region after 1 minute. In contrast, at 1 minute the dye in Region 31 remains a dark blue colored region with a size that has slightly increased as the surface coverage is increased. As shown in FIG. 4D, at 5 hours (18,000 seconds) Region 31 has increased further in size, become more diffuse, and the dark intense center has migrated to a lower portion of the glass. By this time Region 32 has virtually disappeared. This demonstrates the stability of the Trypan Blue layer at the surface of the solution for the 0.3%, yet the 0.06% solution behaves differently dissolving with time.

For reference, if 27 G cannula are used then the solutions are totally mixed within a few seconds, and these mixed solutions remain fully mixed after 5 hours. There is no separation back into a gradient.

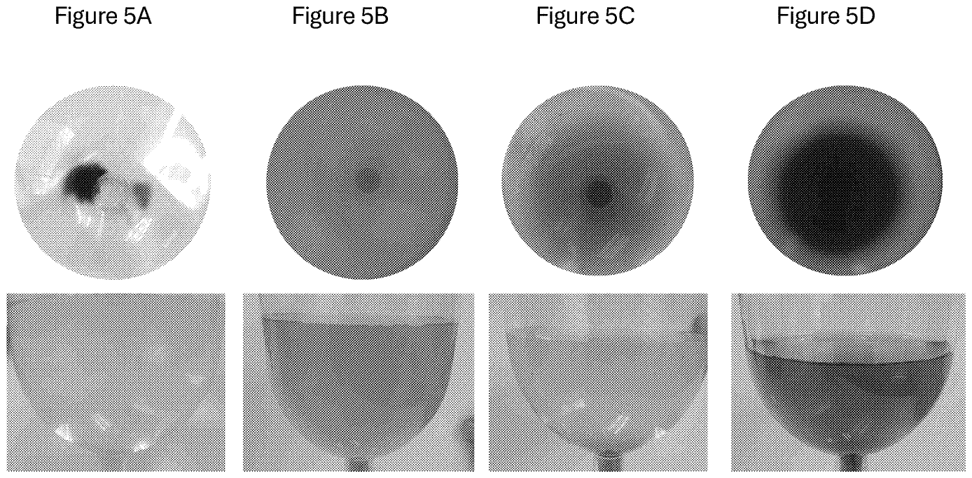

FIGS. 5A, 5B, 5C, and 5D show bird's-eye view images and corresponding side view images comparing Trypan Blue to other colored solutions injected into BSS 60 seconds after delivery: Trypan Blue solution (FIG. 5A), Copper Sulphate solution (FIG. 5B), yellow food dye (FIG. 5C), and blue food dye (FIG. 5D). The Trypan blue forms a thin layer on the base of the glass. Copper Sulphate is a classical ionic solution, and there is no noticeable gradient or surface effects with this solution. Trypan Blue was originally a food dye and has a similar chemical structure to other food dyes. Notably the yellow and blue food dyes more readily dissolved with the fluid in the glass, with a concentrated zone at the base of the glass. Trypan blue is relatively unique in that it is not readily soluble in the BSS if delivered with a 30 G cannula.

The chemical structure of Trypan blue is relatively linear with a medium-length molecular framework. At its center lies an aromatic organic core, comprising two naphthalene rings, which provides structural rigidity and hydrophobic character. Branching out from this aromatic core are two sulfonic acid groups (SO₃H), which act as the polar “heads” located at opposite ends of the molecule. These polar groups make the dye soluble in water and contribute to its overall negative charge in aqueous solutions. Additionally, the molecule is characterized by the presence of azo (—N═N—) linkages, which are key to its vibrant coloration.

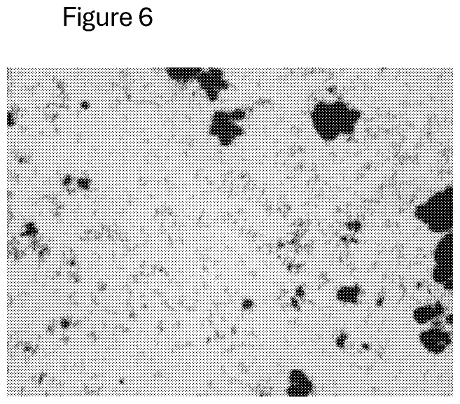

The observations reported here indicated there are some discrepancies with literature. In the literature, Trypan blue is discussed as a polar molecule that is highly soluble. Firstly, Trypan Blue is not so readily soluble. It reaches supersaturated solutions at around 1% concentration. Secondly, Trypan blue is hydrophobic when above critical concentration and prefers self-aggregation but with no specific size, as illustrated by the dark clusters of aggregated Trypan Blue molecules visible in the image shown in FIG. 6. These clusters, which range in diameter up to about 5 microns, formed in a 0.3 % Trypan Blue solution after 0.1 micron filtration of the solution.

Trypan Blue does not have the typical colloidal molecule structure of a polar head and long tail, but it does behave similarly in that it appears to self-assemble in solution when the concentration is above a critical threshold. The differences are that the colloids have a preferred aggregate size i.e., a micelle. In Trypan Blue's case the self-organization is not into sphere micelles, but long chains of weakly bound polymers that clump together, or at surfaces with tissue.

Devices as disclosed herein allow the Trypan Blue concentration to be above the critical threshold, and for the delivery of the solution not to disrupt the weak intermolecular forces between Trypan Blue molecules in the aggregation. This hydrophobic solution has increased efficiencies in staining tissue. Firstly, it can be selectively applied to the tissue rather than broadly flooding the chamber. Secondly, the hydrophobic solution preferentially forms a concentrated layer on the surface. Thirdly, the increased concentration of this distinct solution allows for more rapid dyeing of tissue (diffusion into the membrane).

One embodiment of the invention comprises a blue dye solution with a concentration of 0.2% or higher, in a syringe with about 1 ml capacity, accompanied by a cannula with a smaller internal diameter than a 27 G (210 microns).

Another embodiment of the invention comprises a Trypan Blue dye solution with a concentration of 0.3% to 1%, in a syringe with about 1 ml capacity, accompanied by a 30 G cannula (internal diameter of about 159 microns).

Another embodiment of the invention comprises a 0.3-0.5% concentration by weight Trypan Blue solution, with a volume of 0.25 ml to 0.15 ml in a syringe with a capacity of about 1 ml, accompanied by a 30 G cannula with an internal diameter of about 159 microns.

In these embodiments the volume of the dye solution may be, for example, less than or equal to 0.3 ml, providing ideal ergonomics on the syringe plunger draw positioning.

The cannula may be circular in cross-section. Alternatively the cannula may be non-circular in cross-section to further facilitate application to a larger surface area, such as an oval cross-section with the larger dimension parallel to the plane of the tissue to be dyed. In such a case, the flow rate is governed by the resistance of the cannula average diameter through the length of the cannula. For example, the cannula cross-section may be an oval or flattened oval with an average internal diameter of less than 210 microns, with the preferred average diameter being about 160 microns.

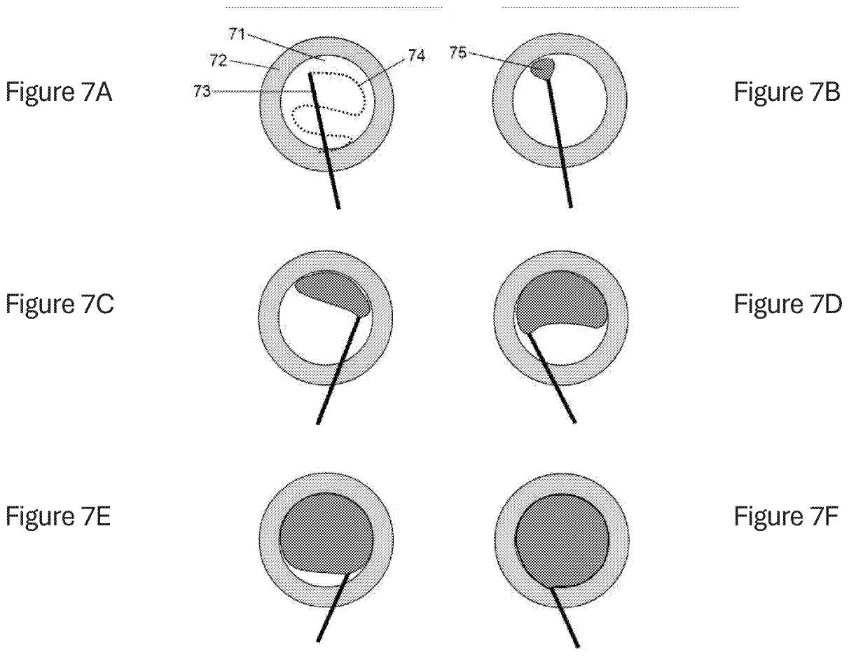

As taught herein, in cataract surgery the preferred device for dyeing the anterior capsule is to use the blue dye solution with a volume of less than or equal to 0.3 ml in a syringe of capacity of about 1 ml and a cannula of less than 27 G to deliver the dye solution with a controllable rate of less than 0.05 ml/s. The preferred method for dyeing the anterior capsule is to apply the distinct cohesive dye stream directly onto or slightly above the anterior capsule, or by filling the pupil well defined by the iris/pupil margin, allowing the cohesive dye stream to pool in the central region over the anterior capsule. One variation comprises painting the anterior capsule using a “zigzag” motion of the cannula distill tip while the dye is being dispensed to cover the target tissue on the anterior chamber as illustrated for example in FIGS. 7A-7F. Ideally the distill tip of the cannula is visible and not submerged in the dye solution stream. The dye is allowed to sit on the capsule for a few seconds, then the excess dye which is not absorbed is washed-out of the anterior chamber with BSS revealing the dyed tissue which is colored by the dye. In this case, the visibility of the anterior capsule is increased for capsulorhexis and for the remainder of cataract surgery including phacoemulsification, Irrigation/Aspiration and IOL insertion.

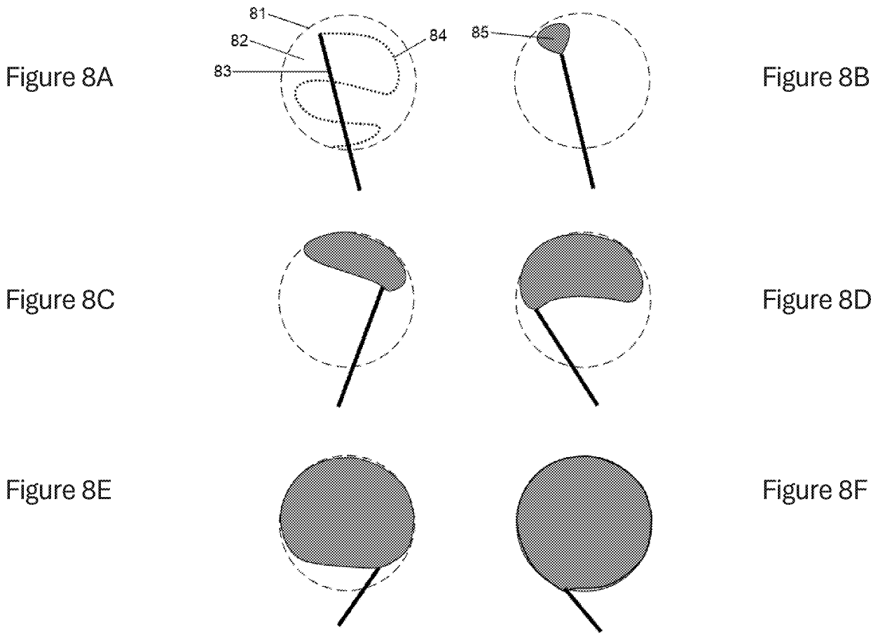

As taught herein, in VR surgery the preferred device for dyeing the anterior capsule is to use the blue dye solution with a volume of 0.3 ml or less in a syringe of capacity of about 1 ml and a cannula of less than 27 G to deliver the dye solution with a controllable rate of less than 0.05 ml/s. The preferred method for dyeing the ILMs or ERMs is to apply the dye directly onto or slightly above the region of the retina where there is interest. The dye is extruded out of the cannula and forms a cohesive pool that is positioned on to this region of the retina and allowed to sit for a few seconds. Then the excess dye which is not absorbed is aspirated out of the posterior chamber with BSS, revealing the dyed tissue which is colored by the dye. The preferred method for applying dye to the retina region of interest is a “zigzag” painting technique of the cannula distill tip while the dye is being dispensed, as shown for example in FIGS. 8A-8D. The tip of the cannula should ideally be visible and not submerged in the cohesive dye solution stream.

The following enumerated clauses provide additional non-limiting aspects of the disclosure.

Clause 1. A device comprising:
a syringe having a volume capacity of about 0.75 ml to about 1.5 ml;
a solution comprising Trypan Blue at a concentration of greater than or equal to 0.2% by weight disposed in the syringe; and
and a cannula having an average inner diameter of less than 210 microns packaged with the prefilled syringe.

Clause 2. The device of clause 1, wherein the syringe has a volume capacity of about 0.9 ml to about 1.2 ml.

Clause 3. The device of clause 1, wherein the syringe has a volume capacity of about 1.0 ml.

Clause 4. The device of any one of clauses 1-3, wherein the Trypan Blue concentration is greater than or equal to 0.25% by weight.

Clause 5. The device of any one of clauses 1-3, wherein the Trypan Blue concentration is greater than or equal to 0.3% by weight.

Clause 6. The device of any one of clauses 1-5, wherein the Trypan Blue concentration is less than or equal to 1.0% by weight.

Clause 7. The device of any one of clauses 1-5, wherein the Trypan Blue concentration is less than or equal to 0.5% by weight.

Clause 8. The device of any one of clauses 1-7, wherein the solution has a volume of 0.30 ml or less.

Clause 9. The device of any one of clauses 1-7, wherein the solution has a volume of 0.25 ml or less.

Clause 10. The device of any one of clauses 1-7, wherein the solution has a volume of 0.20 ml or less.

Clause 11. The device of any one of clauses 1-7, wherein the solution has a volume of 0.15 ml or less.

Clause 12. The device of any one of clauses 1-11, wherein the solution is isotonic or has a tonicity near that of native aqueous humor and has a pH of 6.8-7.6.

Clause 13. The device of clause 12, wherein the solution has a tonicity of about 304 mOsm/kg.

Clause 14. The device of any one of clauses 1-13, wherein the cannula has an average inner diameter of less than or equal to about 0.184 microns.

Clause 15. The device of any one of clauses 1-13, wherein the cannula has an average inner diameter of less than or equal to about 159 microns.

Clause 16. The device of any one of clauses 1-15, wherein the cannula has a circular cross-section.

Clause 17. The device of any one of clauses 1-15, wherein the cannula has an oval cross-section.

Clause 18. The device of any one of clauses 1-15, wherein the cannula has a flattened cross-section.

Clause 19. A device comprising:
a syringe having a volume capacity of about 1.0 ml;
a 0.25 ml to 0.15 ml volume of solution comprising Trypan Blue at a concentration of about 0.3% to about 0.5% by weight disposed in the syringe; and
and a cannula having an average inner diameter of about 159 microns attached to or attachable to the syringe.

Clause 20. A method comprising using the device of any one of clauses 1-19 to apply the Trypan Blue solution to ophthalmic tissue at a rate of less than or equal to about 0.05 ml/s.

Clause 21. The method of clause 20, comprising applying the solution to the ophthalmic tissue by moving a tip of the cannula in a zigzag pattern along the ophthalmic tissue.

This disclosure is illustrative and not limiting. Further modifications will be apparent to one skilled in the art in light of this disclosure and are intended to fall within the scope of the appended claims.

What is claimed is:

1. A device comprising:
a syringe having a volume capacity of about 0.75 ml to about 1.5 ml;
a solution comprising Trypan Blue at a concentration of greater than or equal to 0.2% by weight disposed in the syringe; and
and a cannula having an average inner diameter of less than 210 microns packaged with the prefilled syringe.

2. The device of claim 1, wherein the syringe has a volume capacity of about 0.9 ml to about 1.2 ml.

3. The device of claim 1, wherein the syringe has a volume capacity of about 1.0 ml.

4. The device of claim 1, wherein the Trypan Blue concentration is greater than or equal to 0.25% by weight.

5. The device of one of claim 1, wherein the Trypan Blue concentration is greater than or equal to 0.3% by weight.

6. The device of claim 1, wherein the Trypan Blue concentration is less than or equal to 1.0% by weight.

7. The device of claim 1, wherein the Trypan Blue concentration is less than or equal to 0.5% by weight.

8. The device of claim 1, wherein the solution has a volume of 0.30 ml or less.

9. The device of claim 1, wherein the solution has a volume of 0.25 ml or less.

10. The device of claim 1, wherein the solution has a volume of 0.20 ml or less.

11. The device of claim 1, wherein the solution has a volume of 0.15 ml or less.

12. The device of claim 1, wherein the solution is isotonic or has a tonicity near that of native aqueous humor and has a pH of 6.8-7.6.

13. The device of claim 12, wherein the solution has a tonicity of about 304 mOsm/kg.

14. The device of claim 1, wherein the cannula has an average inner diameter of less than or equal to about 0.184 microns.

15. The device of claim 1, wherein the cannula has an average inner diameter of less than or equal to about 159 microns.

16. The device of claim 1, wherein the cannula has a circular cross-section.

17. The device of claim 1, wherein the cannula has an oval cross-section.

18. The device of claim 1, wherein the cannula has a flattened cross-section.

19. A device comprising:
a syringe having a volume capacity of about 1.0 ml;
a 0.25 ml to 0.15 ml volume of solution comprising Trypan Blue at a concentration of about 0.3% to about 0.5% by weight disposed in the syringe; and
and a cannula having an average inner diameter of about 159 microns attached to or attachable to the syringe.

20. A method comprising using the device of claim 1 to apply the Trypan Blue solution to ophthalmic tissue at a rate of less than or equal to about 0.05 ml/s.
